(19) United States
(12) Patent Application Publication
(10) Pub. No.: US 2005/0239063 A1
(43) Pub. Date: Oct. 27, 2005

(54) Determination of risk factors for cataract by aldose reductase genotype

(76) Inventors: Shao C. Lee, Hong Kong (CN); Maggie C. Y. Ng, Hong Kong (CN); Juliana C. N. Chan, Hong Kong (CN); Julian A.J.H. Critchley, Hong Kong (CN); Christina Parry West, Edinburgh (GB); Clive S. Cockram, Hong Kong (CN)

Correspondence Address: WELSH & KATZ, LTD, 120 S RIVERSIDE PLAZA, 22ND FLOOR, CHICAGO, IL, 60606, US

(21) Appl. No.: 10/473,537

(22) PCT Filed: Mar. 29, 2002
(86) PCT No.: PCT/CN02/00214

§ 371 Date: Jun. 14, 2005

Related U.S. Application Data
(60) Provisional application No. 60/280,529, filed on Mar. 30, 2001.

(51) Int. Cl. C12Q 1/68

(52) U.S. Cl. 435/6

(57) ABSTRACT

This invention relates to methods for determining the risk of developing cataract in mammals, and, more specifically, in mammals, including humans, with non-insulin dependent diabetes. The methods are performed by determining the presence or absence of genetic markers in genomic DNA. The presence of a Z-2 allele, a microsatellite marker of the aldose reductase, is indicative of an increased risk for developing cataract. The presence of Z-4, a microsatellite allele of the aldose reductase gene, indicates a decreased risk for developing cataract. The methods comprise nucleic acid probes that hybridize to a nucleic acid encoding the microsatellite region of the mammalian aldose reductase gene. The probes may also be immobilized on a solid support to form a microarray. In addition, this invention is directed to methods for treating a mammal with a genetic predisposition for developing cataract; following a screening method that detects a Z-2 microsatellite marker of an aldose reductase gene the mammal is treated with an inhibitor of aldose reductase.

### INTRODUCTION

1. Technical Field

The field of this invention is methods and compositions for determining risk of developing cataract in mammals, and further including mammals, including humans, with non-insulin dependent diabetes. The present invention is exemplified by the association of discrete microsatellite markers of the promoter region of the mammalian aldose reductase gene and a genetic predisposition for the development of cataract, particularly in individuals with non-insulin dependent diabetes, including members of the Chinese population with type 2 diabetes. The method includes detecting microsatellite markers Z-2 and Z-4, which comprise alleles of the mammalian aldose reductase gene. Using a nucleic acid sample from an individual being tested, the absence of the Z4 allele or the detection of the presence of the Z2 allele indicates an increased risk for the development of cataract. Conversely, the absence of the Z2 allele or the presence of the Z4 allele in the nucleic acid sample indicates a decreased risk for developing of cataract.

2. Background

The lens of the eye is a proteinaceous structure located near the front of the eye, and is responsible for focusing light and images to the back of the eye and onto the retina. Occasionally, and for reasons which remain largely unknown, some of the lens protein may coagulate over time. This may result in a significant increase in lens opacity and significant or complete loss of vision. These clumped proteins are collectively known as cataracts. Symptoms of cataract include clouded or blurry vision, halo or double vision, and poor dark and color perception. Cataract is the most common cause of visual loss in developed countries.

Surgery and its associated risks remain the only option for cataract patients. Complications arise in a minority of cases. These may include endophthalmitis (intraocular infection), cystoid macular edema (retinal blood vessels leak fluid which accumulates in the macula, causing decreased central vision), retinal detachment (liquid vitreous fluid passes through a fine tear in the retina causing the retina to separate from the back wall of the eye), posteriorly dislocated lens material (lens material dislocating into the vitreous cavity of the eye), and choroidal hemorrhage (acute bleeding in the choroid, or the delicate tissue including blood vessels underlying the retina, and which may lead to significant vision loss).

Because of the risks, surgery is generally not performed until vision loss significantly impaired. Because of the inherent risks associated with cataract surgery and the delay of treatment, non-surgical options for cataract patients would obviously be advantageous, and early intervention, should it become available, would be most desirable. However, there is presently neither pharmacological means to treat developing cataract, nor the means to detect cataract with a high degree of specificity in early stages of the disease, nor the means to screen persons for a predisposition to the disease.

There are encouraging signs from animal studies that certain types of lens opacification can be delayed or prevented, lending credibility to the objective of cataract prevention in humans (Jacob (1999) supra). Research in the pharmacological treatment of cataract is progressing, with a number of proposed treatments being investigated. Particular attention is being paid to secondary cataract associated with diabetes mellitus.

There has been evidence in animal models that the polyol pathway may be involved in the development of secondary cataract associated with diabetes. Aldose reductase catalyzes the NADPH-dependent reduction of hexoses to their polyols, converting D-glucose to sorbitol and galactose to galactitol. Sorbitol does not diffuse readily out of the eye. Fortunately the affinity of aldose reductase for glucose is low, and little sorbitol is produced under euglycemic conditions. In hyperglycemic conditions, however, sorbitol accumulates (Ko, et aL, et al. (1995) Diabetes 44: 727). As the first rate-limiting enzyme in the polyol pathway, aldose reductase has become a target for investigative studies (Lee et al. (1995) PNAS 92: 2780; and Lee et al (1999) FASEB J. 13: 23). Monitoring of the enzyme may provide a diagnostic indicator of the necessity for therapeutic intervention (Cogan et al. (1984) Ann. Intern. Med. 101: 82.). Recently, Ko and coworkers have found that a microsatellite polymorphism at 5′ of the aldose reductase gene was associated with proliferative retinopathy in a group of Hong Kong Chinese patients with early onset Type 2 diabetes (Ko et al., supra). This association has subsequently been confirmed in Japanese (Ikegishi et al. (1999) Life Sci. 65: 2061) as well as South Americans (Olmos et al. (1999) RevistaMedicade Chile 127: 399).

Recent studies have suggested that certain alleles may be useful in determining genetic predisposition for some visual disorders. For example, Ko et al. demonstrated that a dinucleotide repeat polymorphic microsatellite marker at the 5′ end of the aldose reductase gene is associated with early-onset diabetic retinopathy (Ko et al., supra). Microsatellite DNA sequences are often highly polymorphic, and may be good markers for the study of associations between genes and diseases. The distribution of these markers may indicate a genetic predisposition for a particular disease, and may be effectively used when there is a reason to suspect a higher than average likelihood of developing a particular disease, e.g., when a disease has a familial association, or other factors predispose an individual to a genetic disease, e.g., the link between obesity and diabetes.

### RELEVANT LITERATURE

Aldose reductase inhibitors have been shown to be effective in preventing retinopathy, a disease of the retina, at the back of the eye, and cataract, a disease of the lens, or front of the eye, in animal models. The possibility of drug intervention has been investigated by Robison et al. (1995) Invest Ophthalmol Vis Sci 36: 2368, who examined the benefits of sorbinil, an inhibitor of aldose reductase for preventing cataract and diabetic retinopathies, Devamanoharan et al. (1995) J Ocul Pharmacol Ther 11: 527 in which the anti-hypertensive drug verapamil was used to prevent cataract, and Sato et al. (1999) Exp Eye Res 68: 601, in which structurally diverse aldose reductase inhibitors were shown to prevent cataract formation. These studies demonstrated that, once treatments for cataracts are developed, they may be successfully employed. However, all of these studies were conducted with advanced, rather than early forms of cataract, and the subjects were animal models in which cataracts were induced, rather than routinely screened and diagnosed from a general population.

For a discussion of the relationship between the Z-2 allele of the promoter region of the aldose reductase gene and retinopathy (disease of the retina, the structure at the back of the eye comprising photoreceptors, retinal pigment epithelial cells and supporting tissue), see Olmos et al. (2000) Diabetes Res Clin Pract 47: 169, who linked this marker to fast progression retinopathy, and Ko et al., (supra) for the relationship between the Z-2 allele and early onset retinopathy in human patients with non-insulin-dependent diabetes.

### SUMMARY OF THE INVENTION

This invention relates to methods and compositions for determining risk of developing cataract in mammals with non-insulin dependent diabetes by detecting genetic markers associated with the development of cataract in diabetic individuals. The genetic markers include microsatellite polymorphisms of the promoter region of the aldose reductase gene, which have been determined to be associated with cataract, particularly in persons of Chinese descent with Type 2 diabetes, who may be at particular risk for diabetes-related cataract.

Methods for determining individual risk of developing cataract include the steps of obtaining a sample which contains genomic nucleic acid, such as tissue from autopsy or biopsy specimen or a bodily fluid such as a blood sample from a human or a non-human mammal, and contacting that sample with-a pair of nucleotide primers under such conditions, such as those commonly employed in the polymerase chain reaction (PCR), that amplify the DNA sequences of interest. The specifically amplified DNA may then be detected via one of a number of mechanisms used to detect and/or identify DNA, including probe hybridization, in situ hybridization, nuclease analysis, chromatography, autoradiography, fluorography, Southern blot analysis, direct sequencing, restriction enzyme fragment analysis, single-locus DNA profiling, multi-locus DNA profiling, microarray analysis, fragment electrophoretic mobility, or mobility shift assay. Alternatively, particular genes of interest from the genomic DNA of the subject are subjected to restriction fragmentation, and then screened using PCR primer pairs and PCR restriction fragment length polymorphism analysis (RFLP) techniques to identify the presence or absence of a polymorphism of interest, in this case, a Z-2 or Z-4 allele., in the objective of determining the level of risk of the mammal for developing cataract. Methods for treating a mammal identified as having a genetic predisposition for developing cataract with an inhibitor of aldose reductase are also provided. The compositions include nucleic acid probes that hybridize to a nucleic acid encoding an aldose reductase gene, probes, primers for amplification of the Z-2 and/or Z-4 alleles, and microarrays, comprising the probe.

The invention finds use in identifying the level of risk of an individual mammal with non-insulin dependent diabetes of developing cataract, which information can be used in developing a tailored treatment regimen for the mammal, and in the treatment and/or prevention of diabetic complications such as cataracts in individuals identified as having an elevated risk for developing cataract.

### BRIEF DESCRIPTION OF DRAWINGS

FIG. 1 shows the promoter region of the human aldose reductase gene, with (AC)ₙ microsatellite repeats in boldface, and the location of one pair of forward and reverse primer sequences underlined.

FIG. 2A shows an underlined region of FIG. 1, and identified as a forward primer, which may be used to amplify the microsatellite repeat region of the promoter region of the human aldose reductase gene.

FIG. 2B shows an underlined region of FIG. 1, and identified as a reverse primer (shown in FIG. 2B in 5′ to 3′ orientation), which may be used to amplify the microsatellite repeat region of the promoter region of the human aldose reductase gene.

FIG. 2C shows human aldose reductase gene promoter, microsatellite repeat region. AC microsatellite repeats appear in boldface.

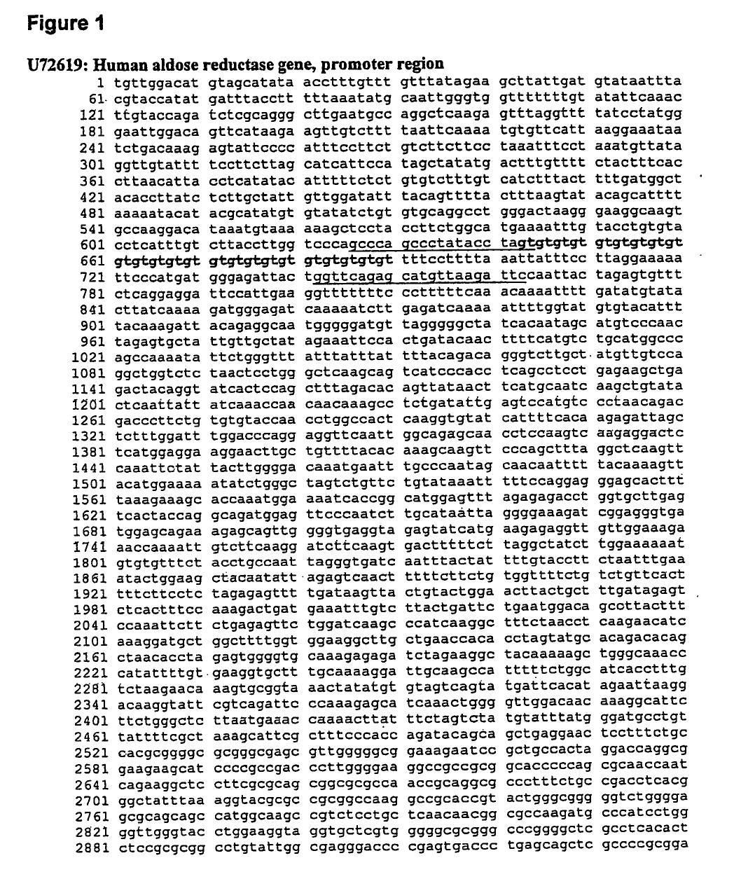

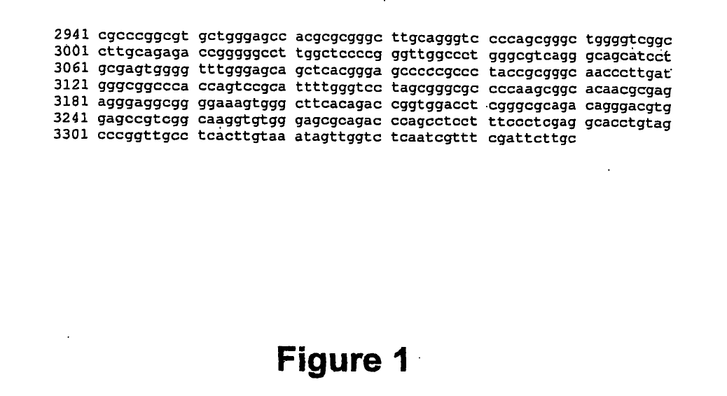

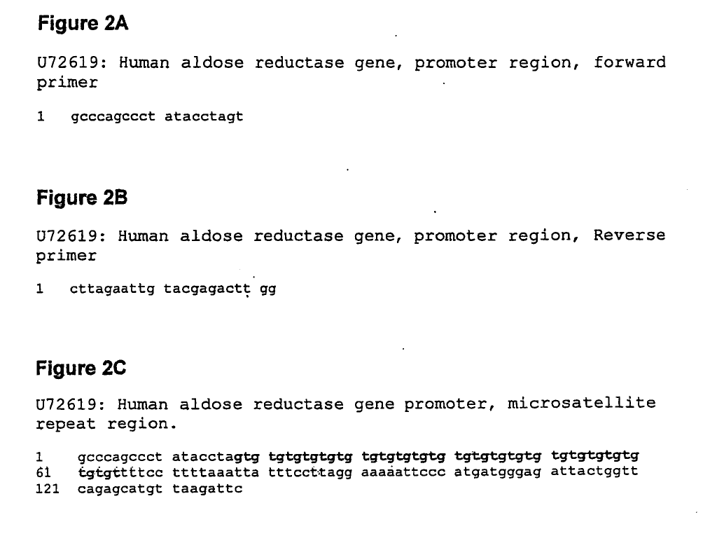

### DESCRIPTION OF SPECIFIC EMBODIMENTS

Compositions and methods are provided wherein microsatellite markers of the aldose reductase gene are used as indicators of risk for developing cataract in mammals with non-insulin dependent diabetes, particularly humans of Chinese descent. The invention comprises as compositions: (1) polynucleotide primers for the amplification of a microsatellite marker of the promoter region of an aldose reductase gene, each primer having a sequence uniquely associated with distinct alleles of an mammalian aldose reductase gene; (2) nucleic acid probes comprising nucleotide sequences that hybridize to the nucleic acid encoding a microsatellite marker of an aldose reductase gene; (3) oligonucleotide probes comprising nucleotide sequences and their complementary sequences; and (4) microarrays of nucleotide probes comprising nucleotide sequences that hybridize to a nucleic acid encoding a microsatellite marker of an aldose reductase gene. The methods involve: (1) obtaining a nucleic acid sample from a mammal; (2) combining the nucleic acid sample with a combination of primers used to amplify the region of genomic DNA of interest, and/or; (3) combining the nucleic acid sample with probes to identify the presence or absence of specific alleles associated with the promoter region of a mammalian aldose reductase gene, by one of a number of methods employed to detect specific DNA sequences. Examples of methods that can be used include in situ hybridization, nuclease analysis, restriction enzyme fragment analysis, chromatography, Southern blot analysis, direct sequencing, single-locus DNA profiling, multi-locus DNA profiling, microarray analysis, fragment electrophoretic mobility, or mobility shift assay. The probes may be attached to a solid support and the solid support may take the form of a microarray.

The phrases “Members of the Chinese population” and “humans/people of Chinese descent” are intended to include individuals of Chinese ancestry. A member of a Chinese population may be more specifically identified by HLA haplotyping. For example, HLA class I and class II frequencies among a Hong Kong Chinese population have been studied by Chang and Hawkins (Hum Immunol (1997) 56: 125). Numerous studies have been carried out to determine HLA class I and class II alleles that are more frequently or even uniquely found in members of a Chinese population, and alleles with strong associations. Shaw et al. and Shen et al. have studied HLA polymorphism and allele frequency and association of Chinese populations in Taiwan (TissueAntigens (1997) 50: 610; Tissue Antigens (1999) 53: 51; JFormos Med Assoc (1999) 98: 11). Allele frequency and associations found in Chinese individuals of mainland China have been reported by Trejaut et al. (Eur J Immunogenet (1996) 23: 437), Shieh, et al (Transfusion (1996) 36: 818), Zhao et al. (Eur J Immunogenet (1993) 20: 293), Wang, et al. (Tissue Antigens (1993) 41: 223; Hum Immunol (1992) 33: 129), Lee, et al. (Eur J. Immunogenet (1999) 26: 275), and Gao et al. (Hum Immunol (1991) 32: 269; Tissue Antigens (1991) 38: 24; Immunogenetics (1991) 34: 401). Additionally, a Chinese individual may be objectively defined by “DNA fingerprinting” techniques well known to those in the art, where microsatellite, short tandem repeat (STR) and variable number tandem repeat (VNTR) loci specific to individuals of Chinese descent are identified. Numerous examples of such ethnic genotyping studies have been reported (Meng, et al. (1999) J Forensic Sci 44: 1273; Yoshimoto, et al. (1999) Int JLegal. Med 113: 15; Wu, et al. (1999) J Forensic Sci 44: 1039; Lee et al. (1999) Eur JImmuhogenet 26: 275; Evett, et al. (1996) Am J Hum Genet 58: 398; Shieh, et al. (1996) Transfusion 36: 818; Gill and Evett (1995) Genetica 96: 69; Balazs (1993) EXS 67: 193; Lan, et al. (1992) Arch Kriminol 189: 169; Fernadez-Vina et al. (1992) Hum Immunol 33: 163; and Hwu, et al. (1992) J Formos Med Assoc 91: 839). All of these above references are incorporated herein by reference. Thus, the risk assessment can include evaluation of the Z-2 and Z-4 alleles with or without evaluation of markers associated with Chinese ancestry.

Advantages of the present invention include that a method for screening would enable early detection of cataract and therefore allow for early initiation of treatment methods, since early initiation of treatment offers the possibility of better prognosis for the individual at risk, since a treatment for cataract is likely to be more effective in the prevention of cataract etiology. Cataract surgery has inherent risks, and is generally not performed until vision loss is significantly impaired. Patients at risk can be screened more vigorously to detect any early changes indicative of developing cataract. Non-surgical options for cataract patients and early intervention would be most desirable. The present screening methods used in conjunction with early detection and treatment of cataract thus would make said detection and proposed treatments more effective in the prevention or early intervention in cataract etiology.

Advantages of the present invention also include that the method of screening uses genetic markers shown to be indicative of either a increased or decreased risk of developing cataract. Methods for screening for genetic markers are well known, and may be configured to be non-invasive, rapid, and of low cost. These methods find particular value when applied to mammals with non-insulin dependent diabetes, including humans with Type 2 diabetes. Identification of the presence or absence of particular polymorphisms in a mammal offers the advantage that with this information health care providers, including physicians and veterinarians, are able to provide more specific and appropriate therapies for individual patients, both human and non-human, and to guide associated therapy and lifestyle adjustments to ameliorate or delay development of cataract, or reduce concern and likelihood of inappropriate medical intervention when there is a decreased probability of cataract development.

Advantages of the present invention additionally include that representative combinations of genetic markers indicative of cataract can be used to screen populations of individuals who may be at increased risk for developing cataract. These individuals may include human patients with type 2 diabetes, so that they may take preventative steps or be given appropriate therapy before the formation or further development of cataract ensues. Family members may also be screened for the particular polymorphisms of the affected individual to identify rapidly family members at increased risk of developing cataract. Advantages of the present invention extend to non-human mammals as well. Dogs and other pets are susceptible to both non-insulin dependent diabetes and cataract. For example, owners of animals diagnosed with non-insulin dependent diabetes can be screened for predisposition to cataract, and the owners counseled regarding the most appropriate treatments and regimens for the animals.

For the screening assays, a sample of genomic DNA is obtained from any nucleated cell source or body fluid. Examples of cell sources available in clinical practice include blood cells, buccal cells, cervicovaginal cells, epithelial cells from urine, fetal cells, or any cells present in tissue obtained by biopsy. Body fluids include blood, urine, cerebrospinal fluid, amniotic fluid, and tissue exudates at the site of infection or inflammation. DNA is extracted from the cell source or body fluid using any of the numerous methods that are standard in the art. It will be understood that the particular method used to extract DNA will depend on the nature of the source. For a further discussion of the isolation of total DNA from tissues, see Rapley and Walker (supra), Chapter 2.

The screening methods may be performed using any of a variety of techniques. The nucleic acid within a sample from the mammal may be specifically amplified prior to the attempt to detect genetic markers associated with increased risk of cataract. As an example, the microsatellite repeat region of the promoter region of the aldose reductase contains specific sequences that provide an indication of the genetic predisposition to cataract. These sequences may be amplified by polynucleotide primers. The PCR primers should be at least 11 base pairs in length, preferably 15-18 base pairs in length, and may be as long as 25-30 base pairs in length. They can be designed to anneal to target regions of DNA including the sequence in regions that flank a gene of interest, or anneal to the gene sequence itself ; in either case extension from the primers amplifies the target region. The invention is exemplified by primer pairs used for the presence of the Z-2 and Z-4 microsatellite alleles of the promoter region of the mammalian aldose reductase gene. Primers can be designed such that their extension results in an amplified sequence only in the presence of either the Z-2 or Z-4 allele, as desired. This can be accomplished by designing a primer with at least one nucleotide at the 3′ end that is mismatched with the Z-2 sequence, but matched to the Z-4 sequence, or vice versa. Of particular interest are nucleic acid primers SEQ ID NO: 1 and SEQ ID NO: 2.

Any one of a number of methods to identify the Z-2 and Z-4 alleles can be used to evaluate the amplified sequence for the presence or absence of the Z-2 and Z-4 alleles of the mammalian aldose reductase gene. As an example, the amplified sequences can be contacted with one or more nucleic acid probes which comprise sequences that hybridize to the nucleotide region encoding the Z-2 and Z-4 alleles of the mammalian aldose reductase gene. The probes may be comprised of DNA or RNA, including oligonucleotides, gene probes and cDNA. The probes may be derived from the promoter region of the aldose reductase gene. They are at least 10 base pairs in length, but can be up to 3350 base pairs in length, the length of the promoter region human aldose reductase gene. The length of the probe chosen will be optimized based on the better base pair mismatch discrimination of shorter probes and the better duplex stability of longer probes (see U.S. Pat. No. 6,156,601 and U.S. Pat. No. 6,197,506, incorporated herein by reference). The length of the probe used should enable discrimination between a mutant and wild-type gene with at least one base-pair mutation. Examples of preferred oligonucleotide probes include SEQ ID NO: 3 and complementary sequences. Methods for preparing probes include synthesis of oligonucleotide probes (Sambrook et al. Molecular Cloning, A Laboratosy MaPlual (2nd Ed), Cold Spring Harbor, 1989, 11.3-11.6) and synthesis of cDNA probes (Sambrook et al., supra, 10.38-10.50) and RNA probes synthesized by in vitro transcription (Sambrook et al., supra, 10.27-10.38), and by plasmid vector (Sambrook et al., supra, 10.29). Probes may also be synthesized de novo (Narang et al. (1980) Methods Enzymol. 65: 610, and Itakura et al. (1984) Ann. Rev. Biochem. 53: 323). Methods for radioactive labeling and hybridization of nucleic acid probes with target sequences on solid supports are discussed in detail in Sambrook et al. (supra) 11.3-11.57, and Keller el al. ((1989) DNA Probes, Stockton, N.Y.). Gene probes, which are generally longer than 500 bases and comprise much of a target gene, may be synthesized according to the methods of Rapley and Walker (Chapter 6 of Molecular Biomethods Handbook (1998) Human Press, Totowa, N.J.). Non-radioactive probes may be prepared according to the methods of Rapley and Walker, Chapter 6 (supra), and as described in the Non-radioactive In Situ Hybridisation Manual (1995), Boerhingher Mannheim GmbH, Mannheim, Germany).).

All of these above references are incorporated herein by reference.

Other useful techniques for identifying the presence or absence of the Z-2 and Z-4 alleles, or other alleles found to be associated with the risk for developing cataract, include single-strand conformation polymorphism analysis (SSCP), denaturing gradient gel electrophoresis (DGGE), fluorescent in situ hybridization (FISH), two-dimensional gel electrophoresis, heteroduplex analysis, dideoxy fingerprinting, enzyme mismatch cleavage (EMC), and others. Detection of known alleles such as Z-2 and Z-4, may be detected using nucleic acid probes in sufficiently stringent hybridization conditions using methods described in the present embodiments.

For SSCP, primers are designed that amplify DNA products of about 250-300 base pairs in length across non-duplicated segments of the gene of interest. For each amplification product, one gel system and two running conditions are used. Each amplification product is applied to a 10% polyacrylamide gel containing 10% glycerol. Separate aliquots of each amplimer are subjected to electrophoresis at 8 W at room temperature for 16 hours and at 30 W at 4° C. for 5.4 hours. These conditions were previously shown to identify 98% of the known mutations in the CFTR gene (Ravnik-Glavac et al. (1994) Hum Mol Gefaet 3:801, incorporated herein by reference).

Nucleic acid probes that hybridize to the specific genetic markers may be arranged on a solid support in multiple discrete regions of distinct nucleic acid strands. This type of array, also known as a microarray, is generally comprised of nucleotide sequences of at least 10 nucleotide bases in length. Such an array comprising nucleotide probes that hybridize to the Z-2 and Z-4 alleles of the aldose reductase gene, or with DNA encoding currently unknown genetic loci that may become associated with risk for developing cataract. The probes arranged on the solid support will generally be comprised of oligonucleotides, and may be generated directly on the solid support or synthesized apart from the solid support and then attached to the support. Alternatively, the probes attached to the solid support may be comprised of homologous sequences of nucleotides isolated directly from the aldose reductase gene or from a DNA library. The array of probes on the solid support may be used as a gene sequencing microarray.

A variety of techniques are then employed to identify the presence or absence of new or known polymorphisms. First, the promoter region of aldose reductase may be sequenced directly using methods that are standard in the art. Polymorphisms may be detected using a PCR-RFLP, procedure, in which pairs of oligonucleotides are used to prime amplification reactions and the sizes of the amplification products, cleaved or uncleaved by restriction endonucleases, are compared with those of control products.

Sequencing microarrays use labeled DNA probes of known, overlapping sequences. Short stretches of DNA are bound to the solid support as probes that differ from adjacent probes by the deletion of a nucleotide from one end and addition of one nucleotide to the other end, (hence, the overlap). The sequence of a DNA fragment from a nucleic acid sample is then determined by hybridization to the overlapping portions under high stringency conditions, which allows only exactly complementary sequences to bind. Thus, the location of hybridizations on the array identifies the complementary sequences that comprise DNA samples. In this manner specific polymorphisms may be detected in a high-throughput manner. This method would find particular use in the screening of a large number of nucleic acid samples. Simultaneous sequencing of several nucleic acid samples can also be carried out on a microarray (see U.S. Pat. No. 6,197,506, incorporated herein by reference).

The microarray generally involves a plurality of different nucleic acid sequences, usually be at least 10, more usually at least 20, frequently at least 50, but may have as many as 100 or more. Microarrays that will find use with the present invention are known in the art (see U.S. Pat. Nos. 5,202,231, 5,741,644, 5,837,832 and 6,183,970, incorporated herein by reference). Additionally, other solid substrates may be used for the covalent attachment of representative combinations of mutated nucleic acid sequences of interest, including beads and slides. Solid supports can be made out of glass or silicon oxide or other materials that can be adapted to be covalently attached to oligonucleotide sequences by the introduction of functionalities which react with oligonucleotides.

A variety of approaches can be used to bind the nucleic acid to the solid substrate. By using chemically reactive solid substrates, one may provide for a chemically reactive group to be present on the nucleic acid, which will react with the chemically active solid substrate surface. For example, by using silicate esters, halides or other reactive silicon species on the surface, the nucleic acid may be modified to react with the silicon moiety. One may form silicon esters for covalent bonding of the nucleic acid to the surface. Instead of silicon functionalities, one may use organic addition polymers, e. g. styrene, acrylates and methacrylates, vinyl ethers and esters, and the like, where functionalities are present which can react with a functionality present on the nucleic acid. For example, amino groups, activated halides, carboxyl groups, mercaptan groups, epoxides, and the like, may be provided in accordance with conventional ways. The linkages may be amides, amidines, amines, esters, ethers, thioethers, dithioethers, and the like. Methods for forming these covalent linkages may be found in U.S. Pat. No. 5,565,324 and U.S. Pat. No. 6,156,501, incorporated herein by reference.

In practice, DNA microarrays can be used to detect both perfectly hybridized and imperfectly hybridized nucleic acids. These microarrays are comprised of a large set of immobilized nucleic acid probes which bind labeled DNA from a nucleic acid sample, which, in itself, generally contains a large set of DNA fragments. These compositions are therefore complex mixtures of sequences. High concentrations of common sequences in a sample increases their degree of hybridization, as well as the fraction of such sequences which are bound to any other sequence. The high local concentration of probe at the surface of a microarray may also trap mismatch binding partners. These effects can reduce both the rate and extent of perfect sequence binding by sequences of average or less than average abundance. Thus, DNA microarrays are imperfect devices, but are quite useful when required for situations where a large number of samples need to be processed rapidly. By determining the best stringency conditions for both hybridization and washing steps through experiment, one may achieve a reasonable level of success in finding conditions which limit binding to that which is near the desired level of sequence matching.

For detection of hybridized probes, including those bound to solid supports, light detectable means are preferred, although other methods of detection may be employed, such as radioactivity, atomic spectrum, and the like. For light detectable means, fluorescence, phosphorescence, absorption, chemiluminescence, and the like can be used. The most convenient is fluorescence, which may take many forms. One may use individual fluorescers or pairs of fluorescers, particularly where one wishes to have a plurality of emission wavelengths with large Stokes shifts. Illustrative fluorescers which have found use include fluorescein, rhodamine, Texas red, cyanine dyes, phycoerythrins, thiazole orange and blue, etc. When using pairs of dyes, one may have one dye on one molecule and the other dye on another molecule which binds to the first molecule. For example, one may have one dye on the first or bound component and the other dye on the second or complexing component. The important factor is that the two dyes when the two components are bound are close enough for efficient energy transfer (see U.S. Pat. No. 5,992,617, incorporated herein by reference).

Single nucleotide polymorphisms (SNPs), preferably but not necessarily occurring within the promoter regions of aldose reductase genes and that correlate with increased risk of cataract, can also be used to evaluate the risk of cataract development. Such SNPs can be identified by correlating polymorphisms in known genes that cosegregate with development of cataract in members of families with a positive history of either cataract or non-insulin-dependent diabetes. SNPs such as those that occur in non-translated and translated regions such as microsatellite promoter regions can be identified through genome-wide scans and correlate linkage analyses of family pedigrees.

As with identification of associative mutations of interest, identification of associative SNPs in alleles that correlate with the risk of a cataract can be accomplished by nucleic acid sequencing of desired regions of genomic or complementary DNA. Screening for SNPs is pursued most efficiently using microarray technologies where attached nucleic acid sequences attached to a solid support such as a microarray are exposed to hybridization conditions that allow the discrimination between two nucleic acid sequences that differ at one nucleotide (see for example, Wang et al. (1998) Science 280: 1077; and Hacia et al (1998) Nature Genet 18: 155). Alternatively, mass spectrophotometers can be used to identify small mass differences in PCR products that have SNPs (see Kirpekar et al. (1998) Nucleic Acids Res 26: 2554; incorporated by reference).

A further means of analyzing SNPs is “dynamic allele specific hybridization” (DASH). This technique uses labeled oligonucleotides in a multiwell format that will fluoresce when the oligonucleotide exists in a double-stranded form, but not when it is in single-stranded form. Adding a single strand of the DNA to be tested allows the strands to hybridize. The temperature at which the strands denature will allow identification of the base at the SNP. The DASH technique has the advantages of being technically simple, and not requiring expensive equipment. Additional techniques that can be used in the screening for SNPs associated with the genetic predisposition of a mamimal to develop cataract include exonuclease resistance, microsequencing, solution-phase or solid phase extension of ddNTPs, and oligonucleotide ligation assay (as described in U.S. Pat. No. 5,952,174). All of the above references are incorporated incorporated herein by reference.

After the presence of an associative mutation or SNP is detected by any of the above techniques, the specific nucleic acid alteration comprising the mutation is identified by direct DNA sequence analysis or restriction analysis or a combination of both. In this manner, previously unidentified SNPs in the aldose reductase gene may be identified.

The invention can be used to assess total risk of developing cataract and/or in designing individualized treatment regimens for diagnosed subjects. The prophylactic screening method may be used for any individual deemed to be at greater risk of developing cataract; i. e., those who may have a family history of cataract, those with diabetes, or those with correlative phenotypic characteristics of diabetes (i. e. obesity). The detection of Z-2 or Z-4 alleles is correlated with phenotypic parameters of screened subjects and may be interpreted with consideration of a positive or negative family history of the disease, or of non-insulin-dependent diabetes. Obtaining a genotypic assessment while a patient shows no signs of developing disease, or while showing preliminary signs of disease, can enable a physician or veterinarian to initiate therapy or suggest changes that prevent the onset or progression of overt symptoms. For humans, treatment can include lifestyle changes such as limiting exposure to sunlight, oxidative stress, cigarette smoke and alcohol, careful attention to treating onset of diabetes, or in its prevention through diet and exercise. Treatments can be initiated early in the disease progression, or before symptoms manifest.

Examples of methods for treating a mammal at risk for developing cataract include detecting the presence of a genetic marker, including the Z-2 microsatellite marker associated with the aldose reductase gene is detected, after which the mammal can be treated with an inhibitor of the aldose reductase gene. Such a treatment is predicated on the activity of aldose reductase as the first rate-limiting enzyme in the polyol pathway. Aldose reductase catalyzes the NADPH-dependent reduction of hexoses to their polyols, converting D-glucose to sorbitol and galactose to galactitol. The enzyme's affinity for glucose is low, and thus not much sorbitol is produced under normal conditions. However, in the presence of hyperglycemia, sorbitol accumulates because it does not readily diffuse out of the eye (Ko et al., supra) causing cataract. Thus, aldose reductase can be used as a target for pharmacological intervention, and inhibitors of this enzyme have been shown to prevent cataract formation in mammals (Robison et al. (1995) Invest Ophthalmol Vis Sci 12: 2368; Devamanoharan et al. (1995) J Ocul Pharmacol Sher 4: 52; and Sato et al. (1999) Exp Eye Res 1999 68: 601). These references are incorporated herein by reference.

Hybridization of nucleic acids with complementary strands is dependent upon a number of chemical and physical factors, including (i) temperature; (ii) salt concentration; (iii) the similarity or “homology” of the hybridizing sequences; and (iv) the concentration of the nucleic acids. The stringency of the hybridization and wash conditions (that is, the physical conditions primarily dependent and salt concentration and temperature) may be used to determine the degree of detection of bound labeled nucleic acid species. This detection may be performed under high stringency conditions, thus limiting detection to sequences that are nearly identical to the labeled sequence, or, by reducing stringency, can be used to detect sequences with lower homology. High stringency of post-hybridization wash conditions may be used to destabilize heteroduplexes that hybridize but contain mismatches (i. e., are below a certain desired degree of homology). In situations where hybridization of sequences that are perfectly homologous to the probe are required, a very high degree of hybridization and wash stringency may be used. High stringency conditions are well known in the art (Ausubel et al. (1989) Current Protocols in Molecular Biology, Vol. I, Green Publishing Associates, Inc., and John Wiley & Sons, Inc., New York, N.Y., at p. 2.10.3, incorporated herein by reference), and include an example of a high stringency hybridization and wash procedure: hybridization to filter-bound DNA in 1% sodium dodecyl sulfate (SDS) and 1 mM EDTA in 5× saline-sodium citrate buffer (SSC) at 65° C., followed by a wash in 0.1×SSC/0.1% SDS at 68° C.

Lower stringency washes may be used to identify sequences that have lower homology to a probe. This could include related alleles. Lower stringency treatments for the purpose of detecting imperfect hybridizations are well known in the art (see, Sambrook et al. supra ; and Ausubel et al., supra, at p. 2.10.3, both incorporated herein by reference). Moderately stringency wash conditions may be achieved by washing in 0.1% SDS in 0.2×SSC at 42° C., and low stringency wash conditions include, 0.1% SDS in 0.2×SSC at room temperature (Ausubel et al., supra, at pages 13.12.1-13.12.5, incorporated herein by reference).

For any given hybridization-based detection mechanism, wash conditions that are required to provide the desired results (i. e., achieve the correct level of detection based on the desired hybridization threshold) must be determined experimentally for each set of sequences of interest.

The methods can be used to screen the genomic DNA of: (i) mammals in general; (ii) mammals with non-insulin dependent diabetes; (iii) humans; (iv) humans with type 2 diabetes, and (v) Chinese persons who have been diagnosed with maturity onset diabetes of the young (MODY) to determine the etiology of their disease, (vi) Chinese individuals that have a positive family history of type 2 diabetes to determine their likelihood of developing diabetic symptoms, and (vii) members of the Chinese population with type 2 diabetes. Polymorphisms associated with cataract are most efficiently identified in Chinese families with a positive history of developing type 2 diabetes (i. e. families with members that develop MODY). However, identified associative polymorphisms are useful for identifying the increased risk in any human, and particularly in a member of a Chinese population.

The following examples are offered by way of illustration of the present invention, not limitation.

### EXAMPLES

Subjects and Diagnosis.

Five hundred sixty seven patients were consecutively recruited at the Diabetes Centre of the Prince of Wales Hospital of Hong Kong, and were diagnosed according to the World Health Organization criteria (World Health Organization Technical Report Series (1985) 727). To increase the homogeneity for Type 2 diabetes, patients who were diagnosed before the. age of 35 years as well as those who were diagnosed for 3 years or less by the time of their the sample recruitment and were already on insulin treatment were excluded. Cataract was evidenced by the presence of lens opacity on direct ophthalmoscopy through dilated pupils and in association with best visual acuity on Snellen's chart≦20/70 in the same eye. The fundoscopy was performed either by ophthalmologists or trained diabetologists.

Clinical, Biochemical and Anthropometric Measurements.

All patients had fasted for at least 8 hour prior to attending the clinical examinations.Blood pressures were measured after they remained seated for at least 5 minutes. Body measurements were taken when patients were standing with light clothing and no shoes. Waist circumference was taken as the minimum circumference between the umbilicus and iliac crest and the hip circumference was taken as the widest circumference around the buttock. Plasma glucose and HbA_{1c} were measured using a glucose oxidase method and an automated ion exchange chromatographic method (normal range: 5.1-6.4%) respectively. Plasma levels of total cholesterol and triglyceride were assayed enzymatically. HDL-cholesterol was determined after fractional precipitation with dextran sulfate-MgCl₂ and LDL-cholesterol, calculated using the Friedewald's equation (Friedewald et al. (1972) Clin. Chem. 18: 499).

Genotyping.

Genotyping for the microsatellite at 5′ of the aldose reductase gene was performed using the primers 5′-GAATCTTAACATGCTCTGAACC-3′ (Sequence ID NO: 1 and 5′-GCCCAGCCCTATACCTAGT-3′ (Sequence ID NO: 2) under the PCR conditions described by Ko and co-workers (Ko et al., supra). Alleles of the microsatellite were electrophoretically separated on polyacrylamide gels and sized against DNA molecular weight standards.

Statistical Analysis.

Continuous variables were expressed as mean ±SD or median (range) as appropriate, which were assessed using Student's t-test and Mann-Whitney Rank Sum test respectively. Chisquare test was performed for analyzing proportions. Multiple logistic regression analysis was performed using cataract as dependent factor (presence=1, not presence=0). Age, sex (male=1, female=0), diabetes duration, fasting plasma glucose, HbA_{1c}, systolic and diastolic blood pressures, body mass index, waist to hip ratio, lipids and the microsatellite alleles Z as well as Z-4 as independent factors. A p value<0.05 was considered to be statistically significant.

Amongst the 567 Chinese patients with Type 2 diabetes, 157 (approximately 28%) had clinical evidence of cataract with visual impairment (s). Compared to the patients without cataract, those with cataract were older in age and age at diagnosis, and had longer diabetes duration (all at p<0.01). The patients with cataract also had higher systolic blood pressures and higher levels of fasting blood glucose (both at p<0.01) and HbA_{1c} (p<0.05) (all with adjustments for the significance) (see Table 1, below).

TABLE 1
Clinical and biochemical features of 567 Hong Kong Chinese
Type 2 diabetic patients with or without cataract

| | Patients with type 2 diabetes | | |
|---|---|---|---|
| | Total | Without cataract | With cataract |
| N | 567 | 410 | 157 |
| Age (years) | 57 ± 12 | 54 ± 11 | 64 ± 10** |
| Sex (M:F) | 1:1.3 | 1:1.4 | 1:1.1** |
| Diabetes duration | 5 ± 5 | 4 ± 4 | 6 ± 6** |
| (years) | | | |
| HbA_{1c}(%) | 7.9 ± 2.0 | 7.8 ± 1.9 | 8.3 ± 2.1** |
| Fasting plasma glucose | 10 ± 3.7 | 8.8 ± 3.5 | 9.5 ± 4.1* |
| (mmol/l) | | | |
| Body mass index | 24.6 ± 3.6 | 24.7 ± 3.4 | 24.4 ± 3.5 |
| (kg/m²) | | | |
| Waist to hip ratio | 0.88 ± 0.06 | 0.88 ± 0.06 | 0.89 ± 0.07 |
| Systolic blood pressure | 136 ± 22 | 134 ± 21 | 143 ± 22** |
| (mmHg) | | | |
| Diastolic blood | 82 ± 11 | 81 ± 11 | 84 ± 11 |
| pressure (mmHg) | | | |
| Total cholesterol | 5.6 ± 1.3 | 5.6 ± 1.3 | 5.7 ± 1.3 |
| (mmol/l) | | | |
| HDL-cholesterol | 1.3 ± 0.34 | 1.3 ± 0.35 | 1.2 ± 0.33 |
| (mmol/l) | | | |
| LDL-cholesterol | 3.5 ± 1.0 | 3.5 ± 1.0 | 3.6 ± 0.9 |
| (mmol/l) | | | |
| Triglyceride (mmol/l) | 1.9(0.38, 26) | 1.9(0.38, 26) | 2.0(0.45, 17) |

Data are expressed in mean ± SD or median (range) as appropriate.
The metabolic parameters were compared with adjustments for age. In addition to age, BMI and WHR were compared with adjustment for sex, and HbA_{1c} and fasting plasma glucose were compared with adjustment for diabetes duration.
*p < 0.05;
**p < 0.01.

A total of 14 alleles of the microsatellite were detected amongst the patients. Allele Z and the Z-carrying genotypes (Z, Z plus Z, non-Z) were over presented in the patients with cataract (both at p<0.01). In contrast, allele Z-4 and its (Z-4, Z-4 plus Z-4, non-Z-4) genotypes were more frequent in the patients without cataract (both at p<0.05) (Table 2).

TABLE 2
Distribution of the major alleles and genotypes of the
microsatellite amongst the 567 Chinese Type 2
diabetic patients with or without cataract.

| Patients with | |
|---|---|
| type 2 diabetes | |
| Without cataract | With cataract |

Allele frequency (%)

| | | |
|---|---|---|
| Z+6 | 3.8 | 4.3 |
| Z+4 | 4.8 | 6.3 |
| Z+2 | 34.4 | 30.0 |
| Z | 22.6 | 29.6** |
| Z−2 | 23.9 | 21.7 |
| Z−4 | 8.3 | 4.8* |
| Others | 2.2 | 3.1 |
| Genotype distribution | | |
| Z⁺, Z⁺:Z⁺, Z⁻:Z⁻, Z⁻ | 9:53:103 | 37:169:204** |
| Z⁺:Z⁻ | 62:103 | 206:204 |
| Z−4⁺, Z−4⁺:Z−4⁺, Z−4⁻:Z−4⁻, Z−4⁻ | 1:24:132 | 0:39:371* |
| Z−4⁺:Z−4⁻ | 25:132 | 39:371 |

*p < 0.05;
**p < 0.01

Using multiple logistic regression analysis, the occurrence of cataract was found to correlate positively with age, but inversely with the presence of the microsatellite allele Z-4 (Table 3). There was no significant differences in mean age between the patients with and those without alleles Z (58±12 vs. 56±12 years) or Z-4 (59±12 vs. 56±12 years).

TABLE 3
Identification of independent risk factors for the development
of cataract in Chinese Type 2 diabetic patients using multiple
logistic regression analysis.

| | β | s.e. | p value |
|---|---|---|---|
| Age | 0.08 | 0.01 | <0.01 |
| Z−4 allele | −0.36 | 0.17 | 0.03 |

R² = 0.23, p < 0.01

Cataract in type 2 diabetes is not uncommon in Caucasian populations. In UKPDS study, the incidence of cataract in newly diagnosed type 2 diabetic patients in different subgroups on a systolic blood pressure scale ranged from 4.7 to 5.2 per 1000 per year (Adler et al. (2000) Brit Med. J. 321: 412). Janghorbani and co-workers followed up their type 2 diabetic outpatients for a mean of 5 years, and observed that the incidences for cataract in their patients with or without insulin treatment were 11.7 and 17.8 per 1000 subjects per year respectively (Janghorbani et al., supra). Amongst the clinic-based cohort of the Hong Kong Chinese patients, 28% had cataracts (Table 1), suggesting that the eye disease is also common in the Chinese type 2 diabetes population. The patients with cataracts appear to be older, with worse glycemic control and higher systolic blood pressure (Table 1). However, age was the only identified independent risk factor (Table 3), which is in keeping with the observations in other populations that, to a large extent, cataract is an aging-related disease (Janghorbani et al., supra, and; McCarty et al. (1999) Am. J. Ophthalmol. 128: 446). There has been evidence that oxidative stress may contribute to the development of cataract (Lee et al. (1994) supra, and; (Lee et al. (1999) supra), and aging increases oxidative stress (Varmaetal, (1995) Critical Rev. Food Nutri. Sci. 35: 111). The association between the occurrence of cataract in Type 2 diabetes and age may thus reflect the effects of oxidative stress (Shah et al. (1998) J Clin. Endocrinol. Metabol. 83: 2886).

In addition to age, the over and under presentations of alleles Z and Z-4 respectively (Tables 2 and 3) implicate that the development of cataract in the Chinese Type 2 diabetes population may be influenced by the genotypes of the aldose reductase gene. This genetic finding is in keeping with the physiological evidence that the polyol pathway is involved in the pathogenesis of diabetic cataract (Lee et al (1995) supra, Lee et al. (1999) supra, and Cogan et al. (1995) supra), and suggests that aldose reductase plays an important role. Aldose reductase catalyses the conversion of glucose into sorbitol. Sorbitol does not easily cross cell membranes and accumulates intracellularly, although it can be physiologically oxidized to fructose by sorbitol dehydrogenase with NAD⁺ as cofactor. Hyperglycemia promotes the influx of glucose through the polyol pathway. This may cause the accumulation of polyol, and result in chronic oxidative and osmotic stress by modifying the availability of NAPDH and NAD⁺, and eventually lead to tissue damages (Lee et al (1995) supra, Lee et al. (1999) supra). Against this background, it is hypothesized that allele Z may be associated with hyperactivity of aldose reductase, which exaggerates the development of cataract. Whereas, allele Z-4 may be associated with hypoactivity of the enzyme, that is protective. There is preliminary evidence that the microsatellite polymorphism may be associated with different expression levels of the aldose reductase gene (Shah et al., supra, and; Ikegishi et al. (1999) Life Sci. 65: 2061). However, more studies are required to clarify the relationship between a specific allele and the gene expression as well as bioactivity of aldose reductase. Moreover, we are aware the observed association between the microsatellite and diabetic cataract may reflect the effect of currently unknown genetic loci that are in linkage disequilibrium with the dinucleotide repeat polymorphism on the development of cataract in the patients.

Summarizing the data, a total of 14 alleles of the microsatellite were detected amongst the patients the Chinese patients with Type 2 diabetes. Allele Z and the Z-carrying genotypes (Z, Z plus Z, non-Z) were over presented in the patients with cataract. In contrast, allele Z-4 and its (Z-4, Z-4 plus Z-4, non-Z-4) genotypes were more frequent in the patients without cataract.

The occurrence of cataract was found to correlate positively with age, but inversely with the presence of the microsatellite allele Z-4. There was no significant differences in mean age between the patients with and those without alleles Z or Z-4.

In conclusion, the results indicate that the development of cataract is common in the Chinese patients with Type 2 diabetes. In addition to age, the aldose reductase gene appears to be an important determinant for this eye disease.

1. A method for determining risk of developing cataract in a mammal, said method comprising the step of:
evaluating a sample comprising nucleic acid from said mammal for presence of at least one of a Z4 allele of the aldose reductase gene and a Z2 allele of the aldose reductase gene wherein at least one of absence of said Z4 allele and presence of said Z2 allele is indicative of an increased risk for developing cataract in said mammal, and wherein at least one of absence of said Z2 allele and presence of said Z4 allele is indicative of an decreased risk for developing said cataract in said mammal.

2. The method according to claim 1, wherein said mammal has non-insulin dependent diabetes.

3. The method according to claim 1, wherein said mammal is a human.

4. The method according to claim 3, wherein said human is of Chinese descent.

5. The method according to claim 1, wherein said evaluating step is performed by contacting said sample with one or more nucleic acid probes, wherein said nucleic acid probes each comprise a sequence that hybridizes to a nucleotide region encoding a microsatellite marker of an aldose reductase gene.

6. The method according to claim 1, wherein said evaluating step is performed by a technique selected from the group comprising:
probe hybridization, in situ hybridization, nuclease analysis, chromatography, autoradiography, fluorography, Southern blot analysis, direct sequencing, restriction enzyme fragment analysis, single-locus DNA profiling, multi-locus DNA profiling, microarray analysis, fragment electrophoretic mobility, or mobility shift assay.

7. A method for detecting increased risk of developing cataract in an individual of Chinese descent having non-insulin dependent diabetes, said method comprising:
amplifying a Z2 allele of an aldose reductase gene in a nucleic acid sample from said individual using primers comprising SEQ ID NO: 1 or SEQ ID NO: 2 whereby amplified sequences are obtained;
hybridizing said amplified sequences under high stringency conditions with a probe comprising a detectable label, wherein said probe comprises a sequence that hybridizes to said Z2 allele; and screening for said detectable label, wherein presence of said Z2 allele is indicative of increased risk of developing cataract.

8. A method for detecting increased risk of developing cataract in an individual of Chinese descent having non-insulin dependent diabetes, said method comprising:
amplifying a Z4 allele of an aldose reductase gene in a nucleic acid sample from said individual using primers comprising SEQ ID NO: 1 or SEQ ID NO: 2 whereby amplified sequences are obtained;
hybridizing said amplified sequences under high stringency conditions with a probe comprising a detectable label, wherein said probe comprises a sequence that hybridizes to said Z4 allele ; and
screening for said detectable label, wherein absence of said Z4 allele is indicative of increased risk of developing cataract.

9. A method for detecting decreased risk of developing cataract in an individual of Chinese descent having non-insulin dependent diabetes, said method comprising:
amplifying a Z2 allele of an aldose reductase gene in a nucleic acid sample from said individual using primers comprising SEQ ID NO: 1 or SEQ ID NO: 2 whereby amplified sequences are obtained;
hybridizing said amplified sequences under high stringency conditions with a probe comprising a detectable label, wherein said probe comprises a sequence that hybridizes to said Z2 allele; and
screening for said detectable label, wherein absence of said Z2 allele is indicative of increased risk of developing cataract.

10. A method for detecting decreased risk of developing cataract in an individual of Chinese descent having non-insulin dependent diabetes, said method comprising:
amplifying a Z2 allele of an aldose reductase gene in a nucleic acid sample from said individual using primers comprising SEQ ID NO: 1 or SEQ ID NO: 2 whereby amplified sequences are obtained;
hybridizing said amplified sequences under high stringency conditions with a probe comprising a detectable label, wherein said probe comprises a sequence that hybridizes to said Z2 allele;
and screening for said detectable label, wherein absence of said Z2 allele is indicative of increased risk of developing cataract.

11. The method according to claim 5, wherein said one or more nucleic acid probes is bound to a solid support.

12. The method according to claim 1, wherein said further comprises the step of contacting said Z-2 or Z-4 allele with a sequencing microarray.

13. A method for treating a mammal in need thereof having an increased risk of developing cataract as a result of presence of a Z-2 allele or absence of a Z-4 allele of aldose reductase, said method comprising:
providing said mammal with an inhibitor of aldose reductase.

14. A primer pair, wherein a first member of said pair comprises SEQ ID NO: 1 and a second member of said pair comprises SEQ ID NO: 2.

15. A polynucleotide primer for amplification of a microsatellite marker of an aldose reductase gene, said primers comprising SEQ ID NO: 1 or SEQ ID NO: 2, or their complementary sequences.

16. A nucleic acid probe wherein said probe comprises a nucleotide sequence of at least 10 nucleotide bases that hybridize to a nucleic acid encoding a microsatellite marker of an aldose reductase gene.

17. The nucleic acid probe according to claim 16, wherein said probe is radioactively labeled.

18. The nucleic acid probe according to claim 16, wherein said probe is labeled non radioactively, and comprise digoxigenin, enzyme, fluorescent, nick-translation, photobiotin, PCR, or an end label.

19. The nucleic acid probe according to claim 16, wherein said nucleotide sequence comprises no more than 3350 bases.

20. The nucleic acid probe according to claim 16, wherein said nucleic acid probe comprises DNA, RNA, or cDNA.

21. An oligonucleotide probe comprising:
a nucleotide sequence of at least 10 consecutive nucleotide units which is included in SEQ ID NO: 3 and its complementary sequence.

22. An array of nucleotide probes comprising:
nucleotide sequences at least 10 nucleotide bases in length that hybridize to a nucleic acid encoding a microsatellite marker of an aldose reductase gene, wherein said nucleic acid comprises an allele that indicates risk for developing cataract, and wherein said array of nucleotide probes is arranged on a solid support in multiple discrete regions of distinct nucleic acid strands.

23. The array of nucleotide probes according to claim 22, wherein said array of nucleotide probes is a sequencing microarray.


SEQUENCE LISTING (3 sequences)

SEQ ID NO: 1 - DNA, 19 bp, Homo sapiens
gcccagccctatacctagt

SEQ ID NO: 2 - DNA, 22 bp, Homo sapiens
cttagaattgtacgagacttgg

SEQ ID NO: 3 - DNA, 138 bp, Homo sapiens
gcccagccctatacctagtgtgtgtgtgtgtgtgtgtgtgtgtgtgtgtgtgtgtgtgtgtgtgttttccttttaaattatttccttaggaaaaattcccatgatgggagattactggttcagagcatgttaagattc